(19) United States
(12) Patent Application Publication
(10) Pub. No.: US 2026/0325758 A1
(43) Pub. Date: Oct. 22, 2026

(54) NEEDLE HUB WITH SPIKE AND OFFSET CANNULA FOR USE WITH A VIAL

(71) Applicant: Koska Family Limited, East Sussex (GB)

(72) Inventors: Marc Andrew Koska, Dartmouth (GB); Max Kingsley Hannon, London (GB)

(21) Appl. No.: 19/653,797

(22) Filed: Apr. 21, 2026

Related U.S. Application Data
(60) Provisional application No. 63/792,858, filed on Apr. 22, 2025.

(51) Int. Cl. A61M 5/28; A61M 5/34

(52) U.S. Cl. CPC A61M 5/282; A61M 5/288; A61M 5/346

(57) ABSTRACT

A medical delivery system comprises a delivery module for coupling to a vial module containing a fluid agent, including a connector body for coupling to the vial, and defining a central longitudinal axis, an administration member coupled to the connector body to distribute the fluid agent from the vial to a patient and a piercing member coupled to the connector body and having a penetrating member configured to access the fluid agent in the vial module. The administration member is arranged about a first axis with the first axis being offset relative to the central longitudinal axis of the connector body.

### BACKGROUND

Staggering numbers of people become infected, severely ill and/or die from a variety of diseases each year, some of which are preventable (or the severity of which could have been mitigated) through injectable medicines, medicaments, vaccines, drug products or other fluid agents (collectively “injectable medicines” herein, injectable medicines referring to fluid agents delivered via use of a syringe or other needle-type medical delivery device). Although injectable medicines have led to a decline in the number of cases of several infectious diseases (or the severity of symptoms or instances of death resulting therefrom) and/or ability to better manage ongoing health conditions such as diabetes, and have also become useful for administration of other medicaments such as contraceptives, the need for additional innovation in injectable medicines remains because the demand for effective contraceptive programs and control of existing and emerging diseases continues to grow and existing solutions leave room for further improvements. Applicant has previously invented various methods and systems intended to address the growing demand for injectable medicines through single-dose, pre-filled blow-fill-seal (BFS) injector devices that comprise one or more benefits, such as (i) being manufacturable in a cost effective manner, in large quantities and at short notice to meet unanticipated peaks in demand; (ii) incorporating simplicity of design that lends itself to administration by users who have little or no medical training (e.g., are suitable for self-injection); and/or (iii) including features that minimize risk of re-use or otherwise prioritize safety concerns. Applicant continues to innovate in this field by developing various new and beneficial systems, devices and components that will provide additional options and opportunities for drug manufacturers to select a single-dose, pre-filled delivery system that best fits their needs.

### SUMMARY

Accordingly, the present disclosure is directed to further improvements and advancements in medical delivery systems including single dose delivery systems. Multiple dose delivery systems are also contemplated.

In accordance with one illustrative embodiment of the present invention, a medical delivery apparatus for coupling to a container containing a fluid agent is disclosed. The medical delivery apparatus comprises a delivery hub for coupling to a container. The delivery hub includes a connector body for coupling to the container where the connector body defines a central longitudinal axis, an administration hub segment for coupling with an administration member to enable distribution of the fluid agent from the container to a patient, and configured to arrange the administrator member about a first longitudinal axis, and an internal penetrating member that is a feature of, or is coupled to, the connector body. In accordance with some embodiments, the internal penetrating member includes a penetrating tip (also referred to as an internal penetrating tip) configured to penetrate the container to access the fluid agent and being arranged about a second longitudinal axis. The second longitudinal axis is different from the first longitudinal axis.

It should be noted that the term “coupled to” (e.g., element A is coupled to element B), as used throughout the present description, is used broadly to encompass configurations in which element A is a feature of a larger element B (e.g., element B is molded such that it includes the feature comprising element A) and also configurations where element A and element B may be molded or otherwise manufactured separately and then linked or arranged so as to cooperatively work together to perform a function. Thus, for example, when an internal penetrating member is described as being “coupled to” the connector body in some embodiments, this encompasses designs in which the internal penetrating member (or “spike”) is a feature of the connector body and the connector body is molded in a single mold process to include the internal penetrating member or spike. In other embodiments, an internal penetrating member may be molded or manufactured separately and then assembled with the connector body, such that the term “coupled to” may encompass embodiments in which two elements are brought together and attached, linked or otherwise assembled to function together.

In embodiments, the administration hub segment defines a bore configured for reception of the administration member where the bore is arranged about the first longitudinal axis.

In some embodiments, the internal penetrating member defines an internal conduit in fluid communication with the administration member.

In other embodiments, the internal conduit of the internal penetrating tip is in fluid coupled relation relative to the bore of administration hub segment.

In embodiments, the internal penetrating tip defines an inlet port in fluid communication with the internal conduit of the internal penetrating member.

In some embodiments, the penetrating tip is configured to extend beyond the inlet port of the internal penetrating member.

In other embodiments, the penetrating tip of the internal penetrating member is beveled.

In embodiments, the internal penetrating member is at least partially disposed within the connector body.

In some embodiments, the internal penetrating member comprises a polymeric material.

In other embodiments, the second longitudinal axis about which the internal penetrating member is arranged is in general alignment with the central longitudinal axis of the connector body.

In embodiments, inner surfaces of the administration hub segment defining the bore include one or more protrusions and one or more recesses.

In some embodiments, the one or more protrusions are configured to engage the administration member.

In other embodiments, the one or more recesses are configured to receive cement or glue used to secure the administration member within the bore.

In embodiments, the medical delivery apparatus includes the administration member which is secured within the bore of the administration hub segment.

In some embodiments, the administration member comprises a needle defining a needle lumen in fluid communication with the internal conduit of the internal penetrating member.

In other embodiments, the needle includes a leading penetrating needle tip.

In embodiments, the medical delivery apparatus further includes a container coupled to the connector body of the delivery hub.

In some embodiments, the container includes a vial having a vial body defining a reservoir for containing the fluid agent and a penetrable seal. The internal penetrating member is configured to penetrate the penetrable seal when the vial is in coupled relation with the delivery hub.

In accordance with another illustrative embodiment of the present invention, a medical delivery system comprises a vial module including a vial having a vial body defining a reservoir for containing a fluid agent and a penetrable seal; and a delivery module. The delivery module includes a connector body for coupling to the vial body, and defining a central longitudinal axis, an administration member coupled to the connector body and having a lumen to permit passage of the fluid agent from the vial to a patient with the administration member being arranged about a first longitudinal axis and a penetrating member coupled to the connector body. The penetrating member defines an internal channel in fluid communication with the lumen of the administration member and has a penetrating tip. The penetrating member is arranged about a second longitudinal axis whereby the second longitudinal axis is radially offset relative to the first longitudinal axis of the administration member.

In embodiments, the second longitudinal axis is in general alignment with the central longitudinal axis of the connector body.

In accordance with one other illustrative embodiment, a medical delivery system includes one or more feature(s) which facilitate access to a container or vial storing a fluid agent, and passage of the fluid agent from the container and through an administration member or needle for delivery to a patient. In some embodiments, the medical delivery system includes an internal penetrating member configured to puncture or penetrate a seal of the container. The internal penetrating member is characterized by having a reduced profile relative to conventional devices. The reduced profile of the internal penetrating member is effected, at least in part, through the arrangement of the internal penetrating member and the administration member which is in fluid communication with the internal penetrating member. In embodiments, the internal penetrating member is in general alignment with respect to a central longitudinal axis of the medical delivery system and the administration member is laterally offset relative to the internal penetrating member. The internal penetrating member further includes an inlet port enabling the fluid agent to directly flow (e.g., generally along a linear path) through a conduit of the internal penetrating member and into the administration member. The inlet port is arranged to permit axial flow and lateral flow of the fluid agent into the conduit of the internal penetrating member. In some embodiments, the inlet port is a single port. In certain embodiments, the combined offset arrangement of the administration member and the internal penetrating member coupled with the inlet port enables the profile of the internal penetrating member to be substantially reduced relative to conventional delivery systems which incorporate multiple access ports within a piercing hub to enable fluid distribution to the administration member. Moreover, due to the multiple access ports and associated configuration of the piercing element, conventional assemblies are relatively large which undesirably increases the profile of these assemblies and presents manufacturing difficulties. In some embodiments, the administration member is a cannulated needle. In certain embodiments, the cannulated needle includes a leading penetrating end. The other trailing end of the needle coupled to the hub may be blunt such that the internal penetrating member penetrates the seal of the container. This eliminates the need for a double-tipped or pencil tipped needle (e.g., cannulated needle with opposed sharp ends) which is relatively more expensive to manufacture, adding more expense in difficulty to manufacture and assemble.

One or more embodiments of the disclosed subject matter provide systems, assemblies, kits, and methods for medical delivery of liquid agents to a patient. Using a blow-fill-seal (BFS) fabrication technique, separate BFS vials (such as bottles, or other components) are pre-filled with respective fluid (e.g., liquid) agents, which can be maintained separate, for example, until ready for use or administration to a patient.

In accordance with another illustrative embodiment of the present disclosure, a medical delivery system comprises a vial module including a vial body defining a reservoir for containing a fluid agent and a penetrable seal, and a delivery module for coupling to the vial module. The delivery module includes a connector body for coupling to the vial body of the vial module and defines a central longitudinal axis, an administration member coupled to the connector body to distribute the fluid agent from the vial module to a patient, and an internal penetrating member coupled to the connector body. The administration member is arranged about a first axis, whereby the first axis is offset relative to the central longitudinal axis of the connector body. The internal penetrating member includes a penetrating tip configured to penetrate the penetrable seal of the vial module to access the fluid agent.

In embodiments, the internal penetrating member defines an internal conduit in fluid communication with the administration member.

In some embodiments, the penetrating tip defines an inlet port in fluid communication with the internal conduit of the internal penetrating member.

In other embodiments, the penetrating tip is configured to extend beyond the inlet port of the internal penetrating member.

In embodiments, the penetrating tip of the internal penetrating member is beveled.

In some embodiments, the internal penetrating member is at least partially disposed within the connector body.

In other embodiments, the internal penetrating member comprises a polymeric material.

In embodiments, the internal penetrating member is arranged about a second axis, whereby the second axis is in general alignment with the central longitudinal axis of the connector body.

In some embodiments, the administration member comprises a needle defining a needle lumen in fluid communication with the internal conduit of the internal penetrating member.

In other embodiments, the needle includes a penetrating needle tip.

In embodiments, the delivery module includes an administration hub whereby the administration member is coupled to the administration hub.

In some embodiments, the penetrating tip of the internal penetrating member of the delivery module is configured to penetrate the penetrable seal of the vial body upon coupling of the vial module to the delivery module.

In other embodiments, the connector body of the delivery module and the vial body of the vial module include cooperating structure to secure the vial module to the delivery module.

In embodiments, the vial body is at least partially compressible.

In some embodiments, the vial module comprise a blow fill seal (BFS) vial.

In other embodiments, the vial module is compressible in one of a longitudinal direction or a lateral direction with respect to the central longitudinal axis.

### BRIEF DESCRIPTION OF THE DRAWINGS

An understanding of embodiments described herein and many of the attendant advantages thereof may be readily obtained by reference to the following detailed description when considered with the accompanying drawings, wherein:

FIG. 1 is an exploded perspective view of a medical delivery system illustrating the vial module and the delivery module in accordance with one or more embodiments of the present disclosure;

FIG. 2 is a side elevation view of the delivery module and the vial module of the medical delivery system in accordance with one or more embodiments of the present disclosure;

FIG. 3 is a side elevation view of the delivery module and the vial module of the medical delivery system in a coupled relation in accordance with one or more embodiments of the present disclosure;

FIG. 4 is a side elevation view of the delivery module of the medical delivery system in accordance with one or more embodiments of the present disclosure;

FIG. 5 is a side cross-sectional view of the delivery module of the medical delivery system in accordance with one or more embodiments of the present disclosure;

FIG. 6 is a cross-sectional view of the connector body of the delivery module of the medical delivery system illustrating the administration hub and the internal penetrating member in accordance with one or more embodiments of the present disclosure;

FIGS. 7A-7C are axial plan, perspective and cross-sectional views of the connector body of the delivery module of the medical delivery system in accordance with one or more embodiments of the present disclosure;

FIG. 8 is an axial view of the delivery module of the medical delivery system in accordance with one or more embodiments of the present disclosure;

FIGS. 9A-9D are views illustrating a sequence of use of the medical delivery system in accordance with one or more embodiments of the present disclosure;

FIG. 10 is a side elevation view of the delivery module and the vial module of the medical delivery system in accordance with one or more additional embodiments of the present disclosure;

FIG. 11 is a side elevation view of the delivery module and the vial module of the medical delivery system in accordance with one or more additional embodiments of the present disclosure;

FIGS. 12A and 12B are side cross-sectional and side plan views of the medical delivery system in accordance with one or more additional embodiments of the present disclosure;

FIG. 13 is a side elevation view of the delivery module of the medical delivery system of FIGS. 12A and 12B in accordance with one or more additional embodiments of the present disclosure;

FIGS. 14A and 14B are side cross-sectional and side plan views of the medical delivery system in accordance with one or more additional embodiments of the present disclosure;

FIG. 15 is a side elevation view of the delivery module of the medical delivery system of FIGS. 14A and 14B in accordance with one or more additional embodiments of the present disclosure;

FIGS. 16A and 16B are side plan and side cross-sectional views of the medical delivery system for delivering insulin in accordance with one or more additional embodiments of the present disclosure; and

FIGS. 17A and 17B are side plan and side cross-sectional views of a conventional prior art delivery system for delivering insulin.

### DETAILED DESCRIPTION

### I. Introduction

Embodiments described herein provide systems and methods for medical delivery devices and/or assemblies that provide significant advantages over current delivery devices and methods. For example, the medical delivery assemblies of some embodiments may include a Blow-Fill-Seal (BFS) vial, bottle or reservoir module which may be coupled to or assembled with a delivery module adapted to distribute the medical agent contained within the reservoir module.

In some embodiments, the vial module may comprise a blow-fill-seal (BFS) vial or container that includes a reservoir (referred to herein as a BFS vial module) prefilled with one or more fluid agents using a BFS manufacturing technique. The BFS vial module may be constructed, filled, and sealed, according to some embodiments, in a sterile manufacturing environment. BFS processes may, for example, offer a less expensive alternative to typical vials or bottles created via other manufacturing techniques. In some embodiments, the BFS vial module (e.g., due to the nature of the BFS manufacturing process) may not require separate sterilization (e.g., and may accordingly be compatible with a wider array of fluid agents), may provide enhanced production rates of sterile/aseptic units per hour, and/or may be provided to an end-user for significantly lower per dose/unit costs. In some embodiments, these advantages may come with attendant drawbacks of reduced manufacturing tolerances and other disadvantages of utilizing a “soft” plastic or polymer (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (November 15, 1989), which is incorporated herein by reference.

In some embodiments, a delivery module can be coupled to the vial module to breach one or more fluid seals thereof with for example, an internal penetrating member, thereby allowing fluid access to the vial module or chamber (e.g., fluid agent can be added to and/or removed from the one or more reservoirs) of the vial module. In some embodiments, the delivery module may include a connector body for coupling to the vial module, an administration member and an internal penetrating member. The administration member is configured to distribute the fluid agent to a patient. In embodiments, the administration member, for example, a cannulated needle, is offset relative to a central longitudinal axis of the connector body. In embodiments, the internal penetrating member is at least partially disposed within the connector body. The internal penetrating member is generally aligned with the central longitudinal axis of the connector body and offset relative to the administration member. The internal penetrating member including an internal penetrating tip configured to access the fluid agent in the vial and defines a lumen or passage in communication with the administration member. The internal penetrating tip includes a port which includes, in embodiments, a lateral or side port segment in communication with the lumen of the internal penetrating member. The internal penetrating member defines a reduced profile due in part at least to the offset arrangement of the internal penetrating member and the administration member and/or the offset side port.

The BFS vial module can have a vial neck that interfaces with the delivery module when the vial neck is inserted therein, which can facilitate coupling of the BFS vial module to the delivery module. In some embodiments, the BFS vial module has a reservoir filled with one or more fluid agents. In some embodiments, the BFS vial module includes a compressible vial body containing the fluid agent and a vial neck extending from the compressible vial body. The vial neck includes a vial neck passage in fluid communication with the reservoir. The penetrable seal is disposed at the end of the vial neck sealing the vial neck passage.

In other embodiments, the vial module may be any conventional vial, container, bottle or reservoir capable of containing a fluid agent, and manufactured by any known manufacturing processes.

In some embodiments, the delivery module includes a connector body couplable to the BFS vial module, a penetrating hub having an internal penetrating member and an administration member, e.g., in the form of a needle. The internal penetrating member pierces or penetrates the penetrable seal upon coupling of the delivery module and the vial module to provide access to the internal vial neck passage and to the fluid agent(s) within the reservoir. In embodiments, the vial module is actuated or compressed whereby the fluid agent(s) is dispensed from the reservoir, through the vial neck passage and into the administration member.

In some embodiments, the administration member comprises a needle or cannula constructed for subcutaneous, intramuscular, intradermal, or intravenous injection of the combined fluid agents into the patient. Alternatively, in some embodiments, the administration member comprises a nozzle. For example, the nozzle can be a spray nozzle that facilitates dispersion of the combined liquid agents into a spray, which configuration may be useful in the administration of the combined fluid agents into a body cavity or orifice (e.g., nasal passage, ear canal, etc.). In another example, the nozzle can be a droplet nozzle that facilitates formation of droplets of the combined fluid agents, which configuration may be useful in the administration of the combined fluid agents to the eyes, for topical application, etc.

In some embodiments, the medical system including the vial module and the delivery module may be coupled to each other on-site and/or in the field. Alternatively, or additionally, in some embodiments, the delivery module may be coupled and/or assembled to the vial module in a manufacturing facility and provided to a user as a single, pre-assembled fluid sub-assembly (e.g., with a vial neck of the reservoir module inserted into a recess of the connector, but without breach of the penetrable seal of the reservoir module).

Embodiments described herein provide systems and methods for pre-filled medical delivery devices and/or assemblies that overcome drawbacks of current delivery devices and methods. For example, the pre-filled medical delivery assemblies of some embodiments may include a delivery module with an internal penetrating member having a reduced profile relative to conventional systems for delivering a medicant. The reduced profile of the penetrating member increases fluid flow through the penetrating member and the cannulated administration member. The reduced profile of the penetrating member simplifies manufacture of the assembly and facilitates coupling of the administration member to the connector body of the delivery module. In other embodiments, the administration member includes a single leading penetrating end with the opposed end coupled to the delivery module being blunt whereby the internal penetrates member provides the seal penetrating capabilities through the seal of the container or vial.

In other embodiments, a medical delivery apparatus is provided without a vial or container, and, in embodiments, without an administration member. The medical delivery apparatus is configured for coupling to a container of any type including glass or hard polymer vials. The medical delivery apparatus may be similar in configuration to the delivery module, including a delivery hub having an internal penetrating member and a hub for coupling to an administration member. The internal penetrating member may include a sharpened tip and may be in alignment with a central longitudinal axis of the delivery apparatus. The hub may include an axial bore for coupling to an administration member at, e.g., the operative site. The axial bore may be arranged about an axis which is radially offset relative to the central longitudinal axis. The internal penetrating member includes an internal conduit in fluid communication with the lumen of the administration member. The internal penetrating member pierces a seal or end of the container when the delivery hub is in coupled relation relative to the container.

Other advantages of the medical delivery assembly will be appreciated by the following description.

### II. Medical Delivery Assemblies

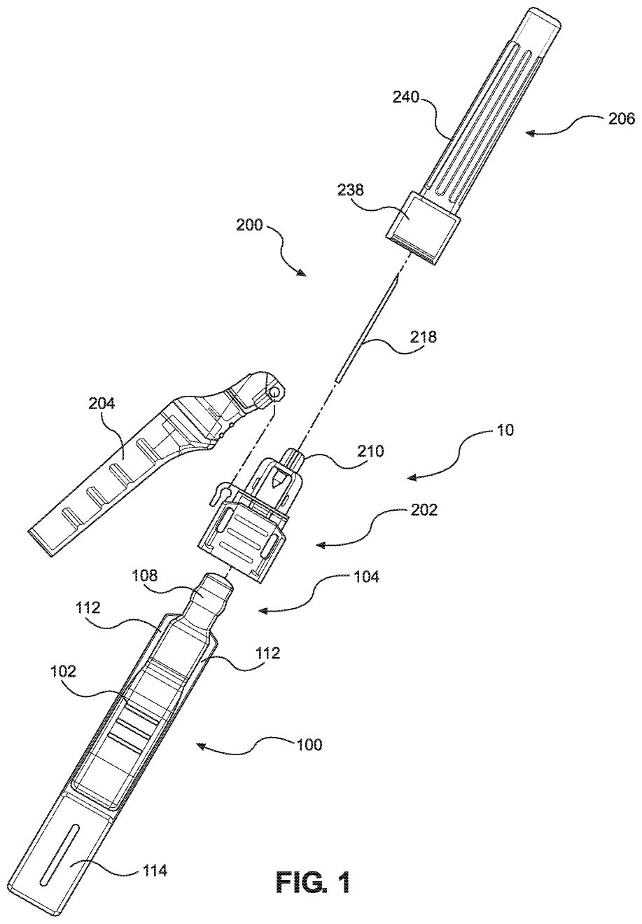

Referring now to FIGS. 1-3, there is illustrated a medical delivery assembly in accordance with one or more embodiments of the subject disclosure. The medical delivery assembly may be prefilled with one or more fluid agents. The medical delivery assembly 10 includes a reservoir or vial module 100 and a delivery module 200 which is couplable to the BFS vial module 100. The vial module 100 may be constructed (e.g., formed) via a BFS process and may comprise a “soft” plastic (e.g., having a Shore/Durometer “OO” hardness of between 60 and 70 and/or a Shore/Durometer “A” hardness between 20 and 50) that is not functionally susceptible to the formation and/or utilization of threaded connection features (as are possible to form on different, harder plastics). During the BFS manufacturing process, the vial module 100 is formed, filled, and sealed in a continuous process without human intervention, in a sterile enclosed area inside a machine. In particular, the BFS manufacturing process involves multiple steps in which a pharmaceutical-grade plastic resin is vertically heat extruded through a circular opening to form a hanging tube called a parison. This extruded tube is then enclosed within a two-part mold, and the tube is cut above the mold. The mold is transferred to the filling zone, or sterile filling space, where filling needles (mandrels) are lowered and used to inflate the plastic to form the container within the mold. Following the formation of the vial module 100, the mandrels are used to fill the vial module 100 with one or more fluid agents. Following filling, the mandrels are retracted and a secondary top mold seals the container. All actions take place inside a sterile shrouded chamber inside the machine. The product is then discharged to a non-sterile area for labeling, packaging and distribution. In association with the BFS process, multiple vial modules 100 are produced and connected to each other as one or more trays or units comprising rows and columns of vial modules 100. Further details of compressible vials are described in international patent application publication number WO 2024/221006, published on 24 Oct. 2024, and entitled “BLOW-FILL-SEAL (BFS) SYRINGE CARTRIDGE SYSTEMS AND METHODS,” the entire contents of the disclosure being incorporated by reference herein.

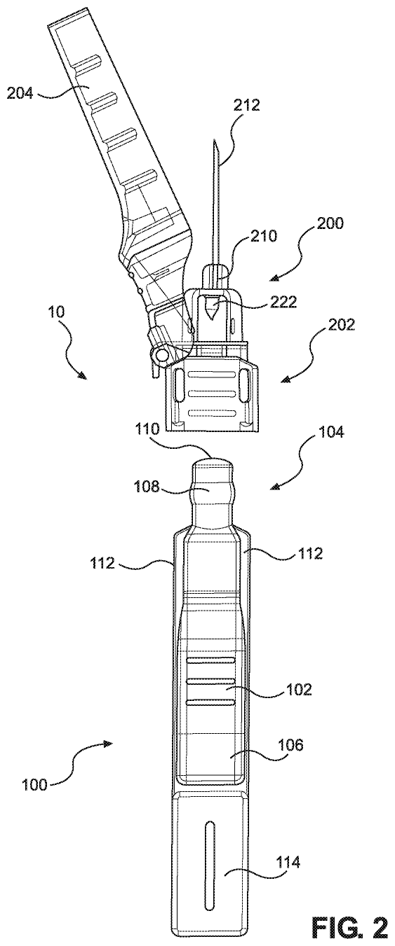

In embodiments, the vial module 100 includes a compressible main vial body 102 and a vial neck 104 extending longitudinally from the vial body 102. In embodiments, the compressible vial body 102 defines an internal fluid chamber 106 (shown in a cut-away in FIG. 2) having one or more fluid agents therein. The vial neck 104 of the BFS vial module 100 defines an internal vial neck passage (not shown) in fluid communication with the internal fluid chamber 106. The vial neck 104 includes a partial or annular outer collar 108 and terminates in a penetrable seal 110. The outer collar 108 may assist in securing the vial module 100 relative to the delivery module 200.

With continued reference to FIGS. 1-3, the vial module 100 may include a pair of diametrically opposed wings 112 depending radially outwardly with respect to main vial body 102 of the vial module 100. The wings 112 may assist in aligning the vial module 100 with respect to the delivery module 200 during coupling, and also may assist in securing the vial module 100 relative to the delivery module 200. The vial module 100 may further include a vial handle 114. The vial handle 114 may be planar and include indicia or an indicator (such as an RFID chip) representative of the type, lot, expiration date of the fluid agent contained within the vial module 100. In embodiments, the vial module 100 may be devoid of the wings 112. The wings 112 and the vial handle 114 may be formed during the BFS manufacturing process. The vial module 100 may comprise protrusions or other irregularities on the outer surface of the vial body 102 to assist in handling of the vial module 100. The vial module 100 may include transparent veins or windows to facilitate viewing within the reservoirs to check the volume of the fluid agents.

In some embodiments, the vial module 100 (e.g., formed via BFS manufacturing processes) may be specifically configured to achieve a squeeze force rating within a pre-defined squeeze force threshold. According to some embodiments, at least the compressible vial body 102 of the vial module 100 may be designed such that a squeeze-force of between thirty newtons (30-N) and forty-five newtons (45-N) is capable of compressing the vial module 100 to such an extent that only a small percentage (e.g., less than twenty percent (20%), less than ten percent (10%), and/or less than five percent (5%)) and/or volume of fluid or medicament remains in the vial module 100. Other squeeze forces for the vial module 100 are contemplated.

The fluid agent may include any type of agent to be injected into a patient (e.g., mammal, either human or non-human) and capable of producing an effect (alone, or in combination with an active ingredient). Accordingly, the agent may include, but is not limited to, a vaccine, a drug, a therapeutic agent, a medicament, a diluent, an active ingredient, and/or the like. According to some embodiments, the fluid agent and the active ingredient (i.e., the drug agent and/or components thereof) may be tracked, monitored, checked for compatibility with each other, etc., such as by utilization of electronic data storage devices (not shown) coupled to the various modules or components such as the vial module 100 and/or the delivery module 200.

In some embodiments, the vial module 100 may be any suitable container, bottle or vial capable of containing a medical agent fabricated by any conventional manufacturing technique. For example, the vial module 100 may be compatible, or integrated, with insulin needle hubs replacing the double needled configuration associated with those assemblies.

Referring still to FIGS. 1-3, the delivery apparatus or module 200 will be described. The delivery module 200 includes several components which are assembled together, e.g., at the factory or a manufacturing facility, or alternatively assembled at the patient site. According to some embodiments, at least some of the components of the delivery module 200 may be composed of a medical grade material. In some embodiments, the components of the delivery module 200 may be composed of a thermoplastic polymer or other “hard” plastic (e.g., greater than 80 on the Rockwell “R” scale), including, but not limited to, polybenzimidazole, acrylonitrile butadiene styrene (ABS), polystyrene, polyvinyl chloride, or the like. The delivery module 200 may include, from proximal to distal, a connector body 202, a pivoting shield 204 coupled to the connector body 202 and a protective cover 206 (shown in FIG. 1).

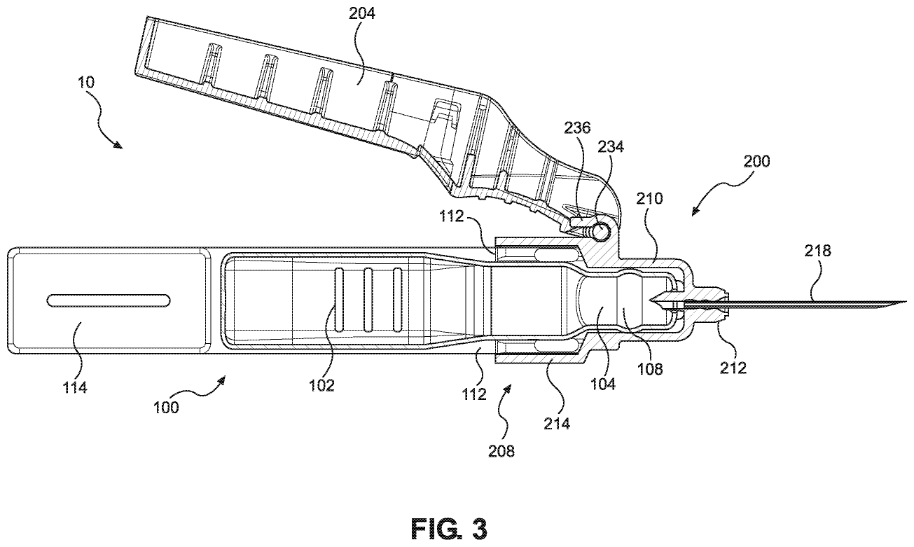

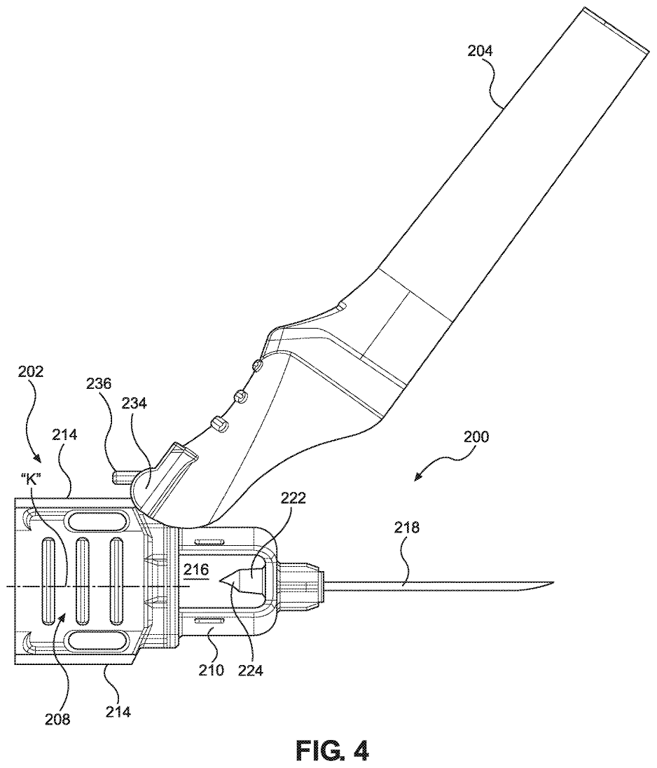

As best depicted in FIGS. 3-5, in conjunction with FIGS. 1 and 2, the connector body 202 includes a main body segment 208, a central hub segment 210 and an administration hub or hub segment 212. The connector body 202 defines a central longitudinal axis “k” (FIG. 4). The main body segment 208 is dimensioned to receive at least a portion of the vial module 100. In embodiments, the main body segment 208 may include outer channels 214 which may be configured to receive the wings 112 of the vial module 100 upon coupling of the vial module 100 to the delivery module 200. In embodiments, the inner surfaces of the outer channels 214 are configured to frictionally engage the wings 112 of the vial module 100 or alternatively deform the wings 112 to assist in retaining the vial module 100 and the delivery module 200 in coupled relation during use. The central hub segment 210 defines an internal hub chamber 216 which receives the vial neck 104 of the vial module 100.

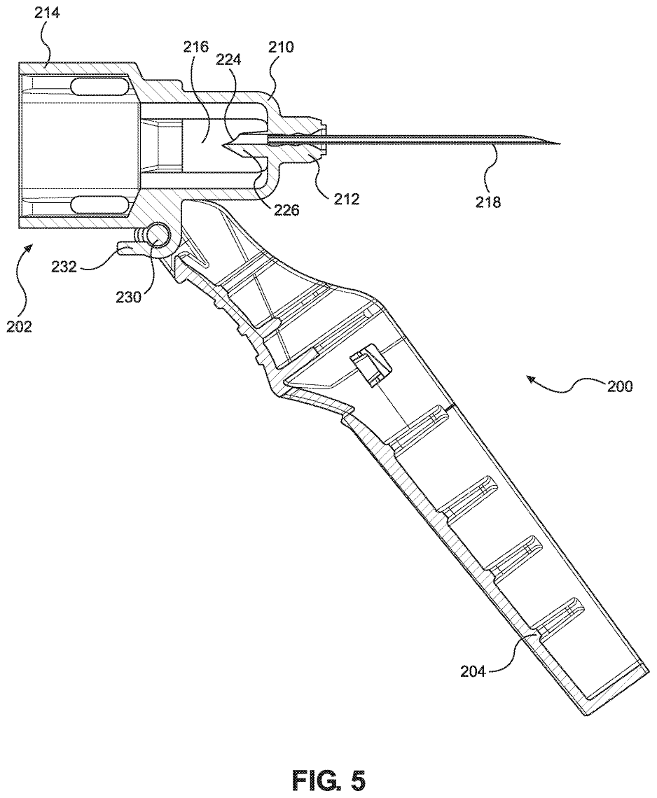

An administration member 218 is coupled to the administration hub 212. The administration member 218 may be a cannulated needle defining a lumen to enable passage of the fluid agent. The leading or distal end of the administration member 218 may be sharp to penetrate the skin of the patient. In the alternative, the administration member 218 may be a dropper or the like. The administration member 218 may be received within a bore 220 (FIG. 5) of the administration hub 212 and secured to the administration hub 212 with adhesives, cements or the like. In some embodiments, the administration member 218 may comprise a needle shaped and/or sized for at least one of subcutaneous, intramuscular, intradermal, and intravenous injection of the fluid agent into the patient. For ease of explanation and description, the figures and the description herein generally refer to the needle as a needle. However, it should be noted that, in other embodiments, the administration member 218 may also or alternatively comprise a nozzle (not shown) configured to control administration of the fluid agent to the patient. The nozzle may comprise a spray nozzle, for example, configured to facilitate dispersion of the fluid agent into a spray. Accordingly, a version of the administration member 218 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a version of the administration member 218 fitted with a droplet nozzle may be useful in the administration of a fluid agent by way of droplets, such as administration to the eyes, topical administration, and the like.

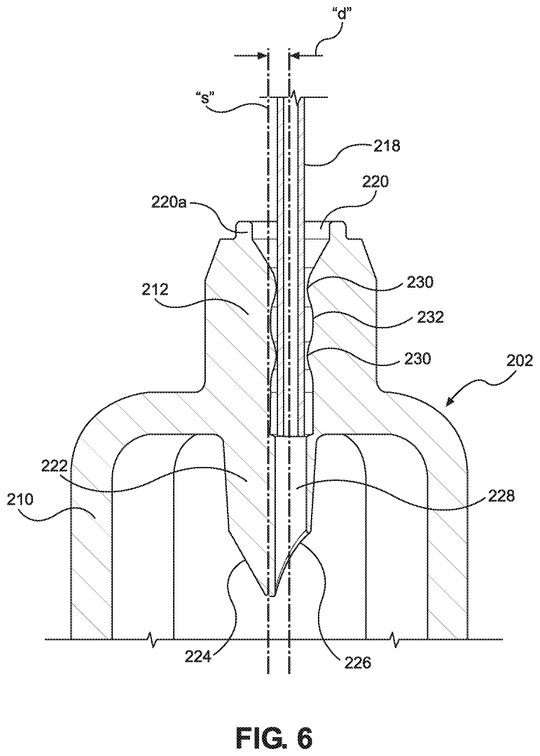

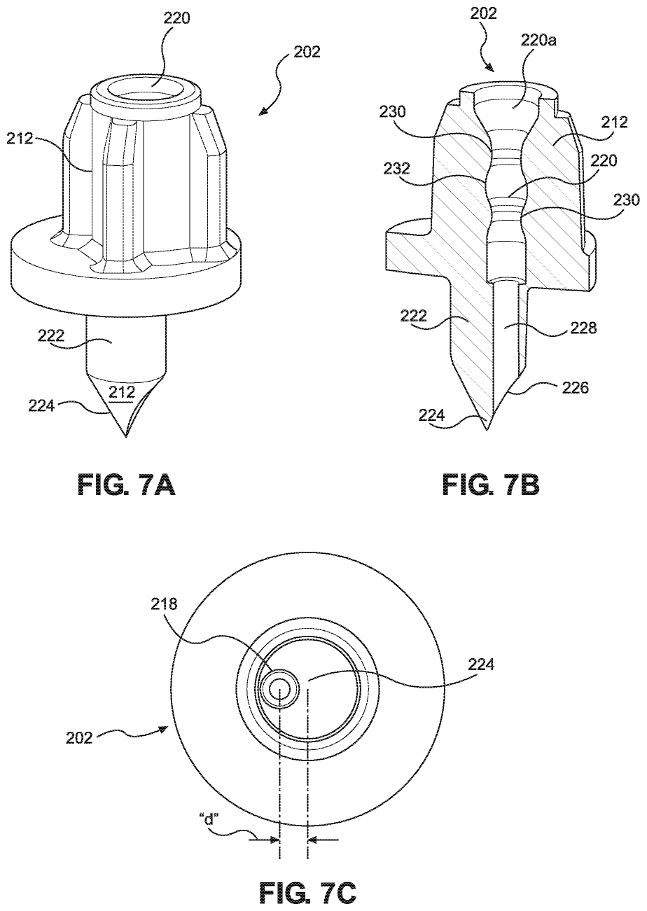

With reference to FIGS. 4-7C, an internal penetrating member 222 extends from the administration hub 212 into the internal hub chamber 216. The internal penetrating member 222 may include a penetrating tip 224 configured to engage the penetrable seal 110 of the BFS vial module 100. As best depicted in FIG. 6, the penetrating tip 224 may be beveled and extend to a solid point. In other embodiments, the point or tip of the internal penetrating tip 224 is open at least in part. The beveled penetrating tip 224 may be configured to penetrate tissue of the patient. The internal penetrating member 222 may include an inlet port 226 leading to an internal conduit 228 (in fluid communication) of the internal penetrating member 222 to enable passage of the fluid agent from within the vial module 100 into the administration member 218. (FIGS. 6 and 7B). In embodiments, the inlet port 226 may be a side or lateral port, an axial port or a combination of an axial port and a side port. In embodiments, the inlet port 226 includes an axial component and a side component, i.e., the inlet port 226 opens in the axial and lateral direction. In embodiments, the penetrating tip 224 extends beyond the inlet port 226. The internal penetrating member 222 may be integrally or monolithically formed with the administration hub 212. In other embodiments, the internal penetrating member 222 may be a separate component coupled to the administration hub 212. In embodiments, the internal penetrating member 222 may define a longitudinal length ranging from about three (3) millimeters (mm) to about five (5) mm or about four (4) mm. Other dimensions are also contemplated.

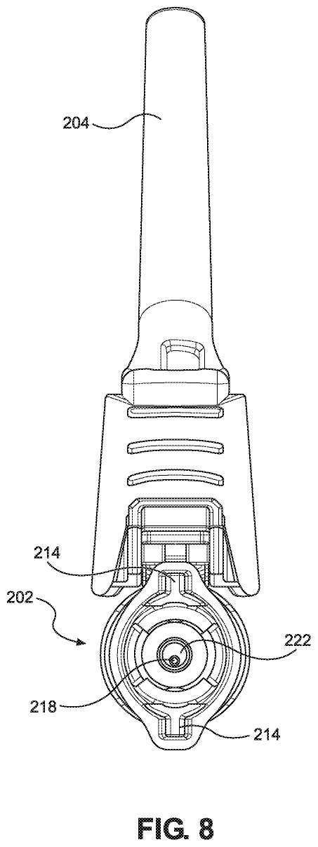

With continued reference to FIGS. 6-8, the administration member 218 is arranged about a first axis “f” and the internal penetrating member 222 is arranged about a second axis “s”. (FIG. 6) The second axis “s” is in general alignment with the central longitudinal axis “k” of the connector body 202. The first axis “f” of the administration member 218 is laterally offset relative to the central longitudinal axis “k” and relative to the second axis “s”. In embodiments, the first axis “f” is laterally offset relative to the central longitudinal axis “k” and relative to the second axis “s” by a distance “d” ranging from about 0.25 mm to about 0.75 mm or about 0.50 mm. (FIGS. 6 and 7C). Other dimensions are also contemplated. This arrangement reduces the profile of the internal penetrating member 222 and the overall profile of the connector body 202. More specifically, the offset profile of the administration member 218 in combination with the configuration of the inlet port 226 (with the axial and side port opening components) of the internal penetrating member 222 enables the fluid agent to directly flow (in, e.g., a longitudinal direction) from the vial module 100 into the internal penetrating member 222 and the administration member 218. In embodiments, only a single inlet port 226 is needed. This is in contrast to conventional arrangements requiring multiple openings or ports through which a fluid must laterally enter for passage out a needle or the like. Offsetting the administration member 218 from the central longitudinal axis “k” allows the central connector body 202 to incorporate a properly sized bore 220 as, in embodiments, a glue channel to secure the administration member 218. By shifting the inlet port 226 of the internal penetrating member 222 to one lateral side while keeping the penetrating tip 224 of the internal penetrating member 222 centered with respect to the central longitudinal axis “s”, the size of the bore 220 is optimized to form and receive sufficient glue (within, e.g., a glue channel) to properly secure the administration member 218. In embodiments, the administration hub 212 also is arranged about the first axis “f”.

As noted, the second axis “s” about which the internal penetrating member 222 is arranged is in general alignment with the center of the penetrable seal 110 of the vial module 100 when the vial module 100 and the delivery module 200 are coupled. This facilitates penetration of the internal penetrating member 222 through the penetrable seal 110 in that the penetration forces are focused at the center (e.g., direct center) of the penetrable seal 110.

As depicted in FIGS. 6 and 7B, the a bore 220 of the administration hub 212 may define a conical or frusto-conical leading end segment 220a configured to facilitate insertion of the administration member 218. In addition, in some embodiments, portions of the inner surface of the administration hub 212 defining the bore 220 may include one or more protrusions 230 and one or more recesses 232 extending along the central longitudinal axis “k” in, in embodiments, alternating arrangement. The one or more protrusions 230 may be configured to engage the outer surface of the administration member 218 to engage the administration member 218. In some embodiments, the one or more protrusions 230 frictionally engage the administration member 218. In other embodiments, the one or more protrusions 230 may center the administration member 218 within the bore 220. The one or more recesses 232 may receive glue during the bonding process of the administration member 218 within the bore 220.

In embodiments, the internal dimension of the lumen of administration member 218 ranges from about 0.50 mm to about 0.9 mm or about 0.7 mm. Other dimensions are contemplated to ensure adequate distribution through the administration member 218.

With reference again to FIGS. 3 and 4, the pivoting shield 204 includes a pivot bar 234 which snaps into cooperative pivot structure or recess 236 of the connector body 202 to couple the pivoting shield 204 relative to the connector body 202. The pivoting shield 204 may pivot about the pivot bar 234 between an open position displaced relative to the administration member 218 (FIG. 3) and a closed position covering the administration member 218 (with the protective cover 206 removed). In some embodiments, the pivoting shield 204 may include an internal locking latch (not shown) which may be configured to selectively secure the administration member 218 when the pivoting shield 204 is pivoted to cover the administration member 218. In some embodiments, the locking latch may prevent reuse of the administration member 218, i.e., the pivoting shield 204 cannot be pivoted off the administration member 218 once secured to the administration member 218 without deforming and/or breaking the locking latch and/or the administration member 218.

As best depicted in FIG. 1., the protective cover 206 includes a cover housing 238 and an elongated cover component 240 depending from the cover housing 238. The cover housing 238 includes inner wall portions defining an internal cover chamber configured to fit over the central hub segment 210 and the administration hub 212 whereby, for example, an interference fit with the exterior of the central hub segment 210 and the cover housing 238 is established between the components. The elongated cover component 240 encloses the administration member 218 during transport and prior to use. In some embodiments, the protective cover 206 remains connected to the central hub segment 210 during coupling of the delivery module 200 to the vial module 100. Once coupled, the protective cover 206 may be removed.

In some embodiments, the pre-filled medical delivery assembly 10 may be advantageously manufactured (in mass quantities) in separate parts or portions, namely, at least the “soft” plastic vial module 100 (e.g., a “first” piece) and the “hard” plastic delivery module 200 (e.g., the “second” piece), with such different plastic parts/portions being selectively coupled to administer a medication to a patient.

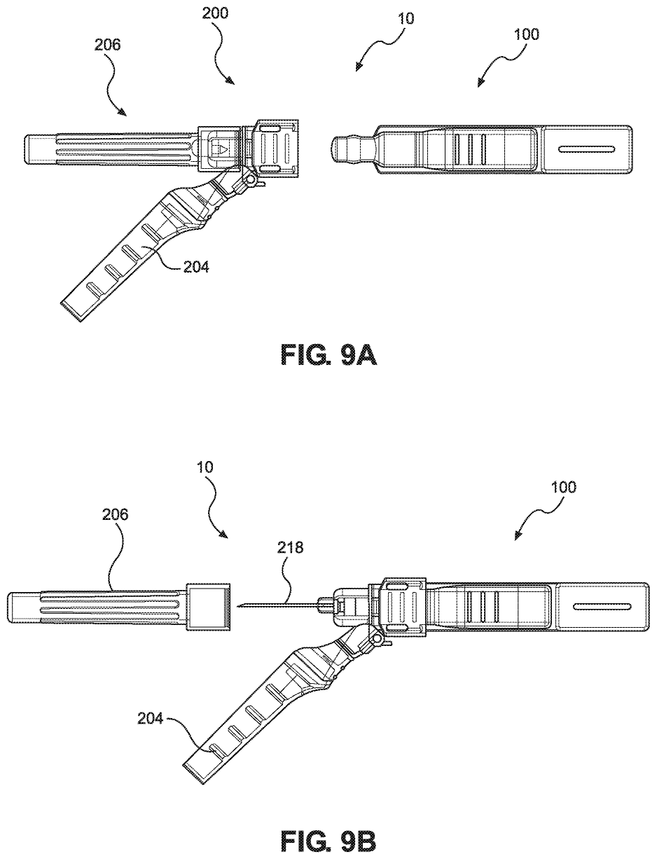

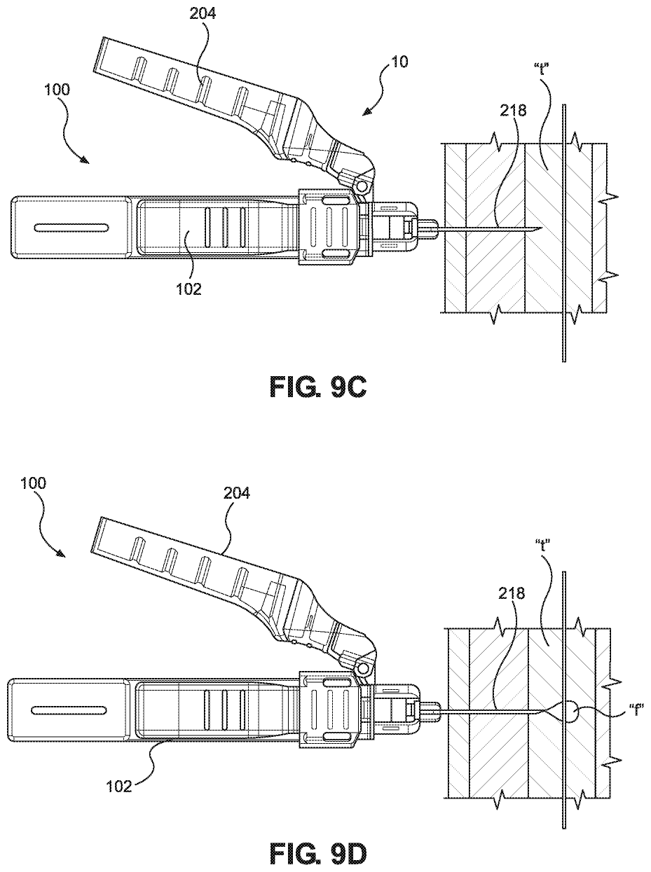

FIGS. 9A-9D illustrate a sequence of use of the medical delivery assembly 10 of the present invention. With reference to FIG. 9A, the vial module 100 and the delivery module 200 with attached protective cover 206 are distributed (independently or together) to the operative or treatment site. The delivery module 200 is longitudinally aligned with the vial module 100 such that the wings 112 of the vial module 100 are aligned with the outer channels 214 of the connector body 202 of the delivery module 200. In embodiments, the internal dimension of the connector body 202 of the delivery module 200 and the wings 112 of the vial module 100 are arranged whereby the vial module 100 may only be introduced within the connector body 202 in the proper orientation with the wings 112 aligned with the channels 214 of the delivery module 200. The vial module 100 is advanced into the connector body 202. During insertion of the vial module 100, the wings 112 may deflect inwardly. Upon continued entry of the vial module 100 into the connector body 202, the wings 112 enter the outer channels 214 of the connector body 202 and are compressed and/or frictionally engage the inner surfaces of the outer channels 214 thereby securing the vial module 100 relative to the delivery module 200. In other embodiments, the annular collar 108 on the vial neck 104 engage cooperative structure of the connector body 202 to also assist in maintaining the vial module 100 and the delivery module 200 in coupled relation.

In addition, during advancement of the vial module 100 relative to the delivery module 200, the internal penetrating member 222 penetrates the penetrable seal 110 of the vial module 100 via the penetrating tip 224 of the internal penetrating member 222. The internal penetrating member 222 accesses the fluid agent within the internal fluid chamber 106 of the vial module 100. The protective cover 206 may be removed as shown in FIG. 9B.

With the internal penetrating member 222 accessing the internal fluid chamber 106, the administration member 218 is advanced within the tissue “t” as shown in FIG. 9C. A squeezing action is exerted on the compressible vial body 102 of the vial module 100 to cause the fluid agent from the vial module 100 to enter the inlet port 226 of the internal penetrating member 222 and pass through the internal conduit 228 of the internal penetrating member 222. The fluid agent “f” is then distributed through the administration member 218 for application to the patient (FIG. 9D). Once delivery of the fluid agent is complete, the pivoting shield 204 may be pivoted over the administration member 218, and, in embodiments, secured to the administration member 218 via an internal latch.

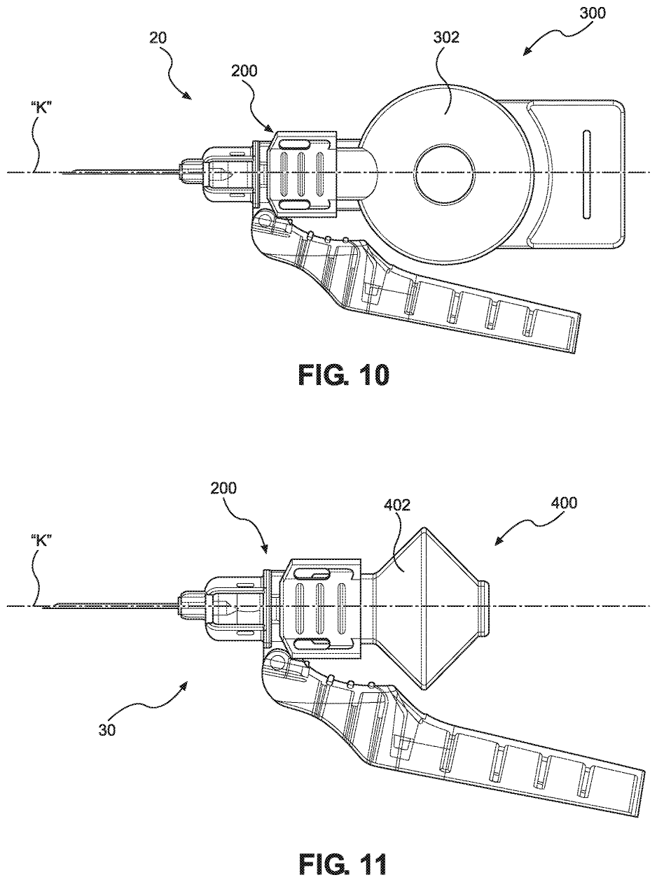

FIGS. 10 and 11 illustrate alternate embodiments of the medical delivery assembly of the present invention. The medical delivery assembly 20 of FIG. 10 includes a vial module 300 and a delivery module 200. The delivery module 200 is similar or identical to the delivery module 200 described hereinabove. The vial module 300 includes a compressible vial body 302 which is compressed in a lateral direction relative to the central longitudinal axis “k” to dispense the fluid agent. The medical delivery assembly 30 of FIG. 11 includes a vial module 400 and a delivery module 200 which is similar or identical to the delivery module 200 described hereinabove. The vial module 400 includes a compressible vial body 402 which is compressed in an axial direction relative to the central longitudinal axis “k” to dispense the fluid agent. In other respects, the vial modules 300, 400 are similar to the vial module 100 of FIGS. 1-9D. The compressible vial bodies 302, 402 of the vial modules 300, 400 may include a bellows structure or the like.

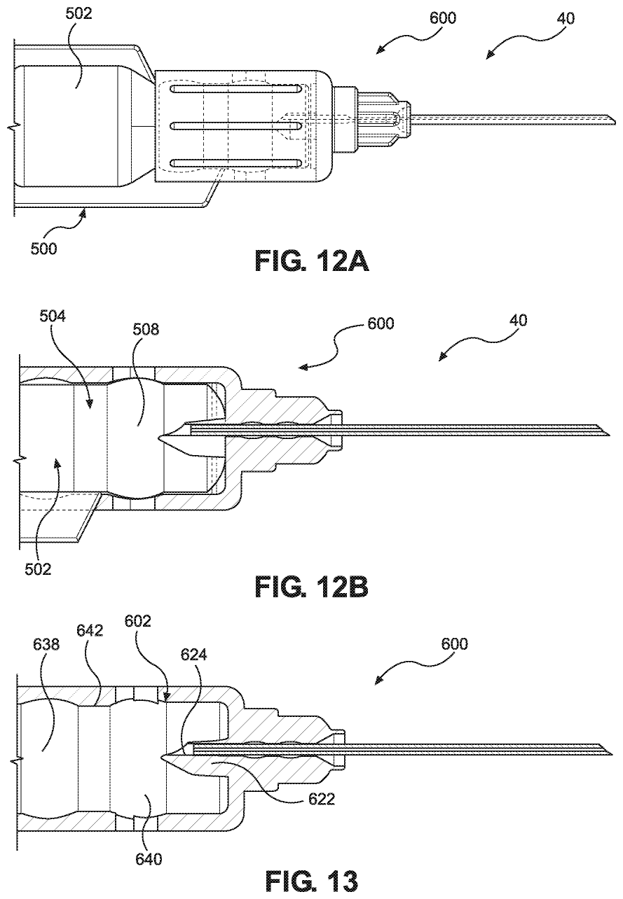

FIGS. 12A, 12B and 13 illustrate alternate embodiments of the medical delivery assembly of the present invention. The medical delivery assembly 40 includes a vial module 500 and a delivery module 600. FIGS. 12A and 12B show the vial module 500 and a delivery module 600 in an assembled condition. The vial module 500 is substantially similar to the vial module 100 of FIGS. 1-11. Only a portion of the vial module 500 is depicted in the figures for illustrative purposes. The delivery module 600 includes a connector body 602 with at least one, and, in embodiments, two internal annular recesses 638, 640 best shown in FIG. 13. Each recess 638, 640 is configured to receive the annular outer collar 508 of the vial neck 504 extending from the main vial body 502 of the vial module 500. In embodiments, each internal annular recess 638, 640 may at least partially compress the outer collar 508 of the vial neck 504. In some embodiments, the outer collar 508 is at least partially compressed when the vial module 500 is at least partially positioned within the delivery module 600 and then returns toward its original configuration once received within the respective internal annular recess 638, 640. In embodiments, the trailing or proximal recess 638 is used to maintain the vial module 500 and the delivery module 600 in an assembled unactuated condition during transport or the like. When in this assembled transport condition, the outer collar 508 of the vial module 500 is received within the proximal recess 638, and the penetrable seal 510 of the vial module 500 is spaced from the penetrating tip 624 of the internal penetrating member 622 of the delivery module 600. When the medical delivery assembly 30 is to be used, the vial module 500 is further advanced into the connector body 602 of the delivery module 600 whereby the outer collar 508 of the vial module is compressed by internal transition section 642 of the delivery module 600, and then returns toward its initial configuration received within the leading annular recess 640 of the delivery module 600. During this movement, the internal penetrating member 622 penetrates the penetrable seal 510 of the vial module 500 for distribution of the fluid agent in the manner described hereinabove. (FIG. 12B). The internal transition section 642 of the delivery module 600 defines an inner dimension or diameter less than the corresponding internal dimensions or diameters of the internal annular recess 638, 640, and less than the outer dimension or diameter of the outer collar 508 of the vial module 500. In embodiments, once actuated the internal transition section 642 of the delivery module 600 may restrict or prevent removal of the vial module 500 relative to the delivery module. In some embodiments, the internal transition section 642 provides a tactile and/or audible indicator, perceptible by the user, that the outer collar 508 is received within the leading or distal annular recess 640 corresponding to the assembly being activated. In other respects, the vial module 500 and the delivery module 700 are substantially similar to the vial module 100 and the delivery module 200 of FIGS. 1-11.

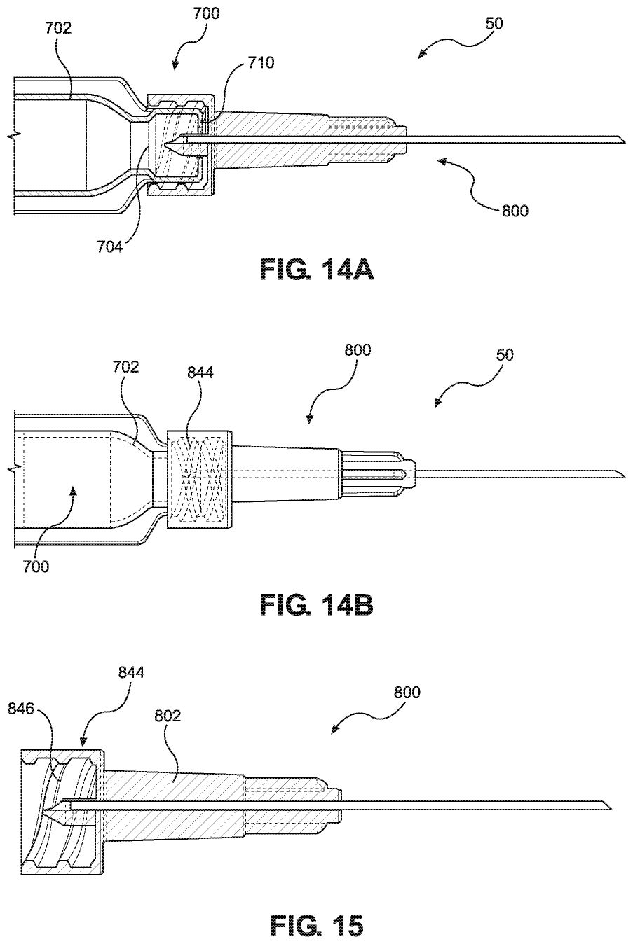

FIGS. 14A, 14B and 15 illustrate alternate embodiments of the medical delivery assembly of the present invention. The medical delivery assembly 50 includes a vial module 700 and a delivery module 800. FIGS. 14A and 14B show the vial module 700 and a delivery module 800 in an assembled condition. The vial module 700 is substantially similar to the vial module 100 of FIGS. 1-11. Only a portion of the vial module 700 is depicted in the figures for illustrative purposes. The vial module 700 includes a vial neck 704 extending from the vial body 702 to a penetrable seal 710. The vial neck 704 includes an at least partial thread 716 on its external surface. The delivery module 800 includes a connector body 802 having a connector hub 844. The connector hub 844 includes an internal partial thread 846 configured to cooperate with the at least partial thread 716 of the vial module 700 to couple the vial module 700 to the delivery module 800. In embodiments, the at least partial threads 716, 846 of the vial module 700 and the delivery module 800 establish a luer coupling or connection-type fitting. When the medical delivery assembly 40 is to be used, the vial module 700 is at least partially positioned within the connector hub 844 of the connector body 802. The vial module 700 is rotated relative to the delivery module 800 whereby the at least partial threads 716, 846 of the vial module 700 and the delivery module 800 cooperate to advance the vial module 700 relative to the delivery module 800 to cause penetration of the penetrating tip 822 of the internal penetrating member 824 pass through the penetrable seal 710 of the vial module to access the fluid agent. In other respects, the vial module 500 and the delivery module 700 are substantially similar to the vial module 100 and the delivery module 200 of FIGS. 1-11.

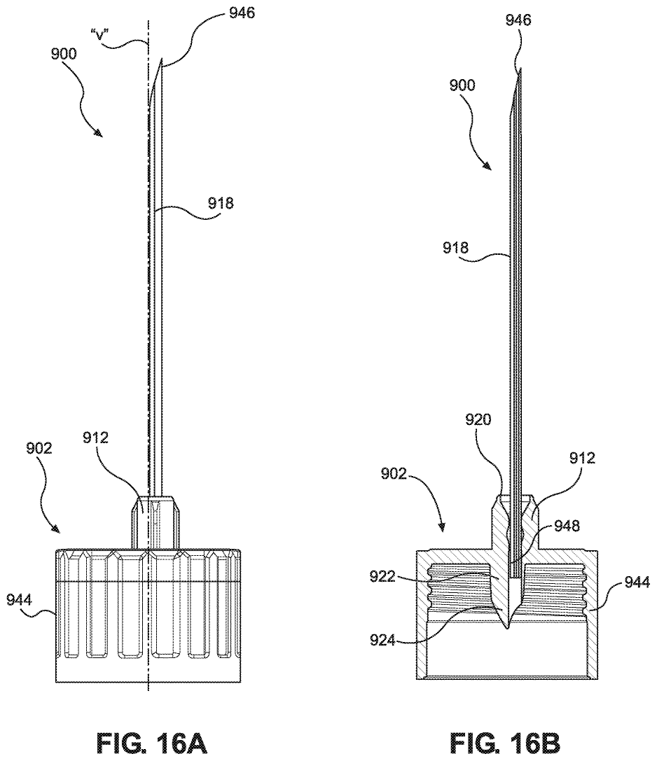

FIGS. 16A and 16B illustrate another illustrative embodiment of the present invention. In embodiments, the delivery module 900 is configured for coupling to a vial or container containing insulin for delivery of insulin to the patient. The delivery module 900 includes a connector body 902 having an internal penetrating member 922 with a penetrating tip 924. The internal penetrating member 922 is aligned with the central longitudinal axis “v” of the delivery module 900 as described in connection with the embodiments of FIGS. 1-15. An administration member 918 is coupled to the administration hub 912 of the connector body 902. The administration member 918 may be a cannulated needle defining a lumen to enable or permit passage of the fluid agent. The leading or distal end 946 of the administration member 918 is sharp (including a bevel shape or the like) to penetrate the skin of the patient. The administration member 918 may be received within a bore 920 of the administration hub 912 and secured to the administration hub 912 with adhesives, cements or the like. In embodiments, the trailing or proximal end 948 of the administration member 918 is flat or blunt. The administration hub 912 and the administration member 918 are offset relative to the central longitudinal axis “v” of the delivery module 900 as described in connection with the embodiments of FIGS. 1-15. The connector body 902 includes a connector hub 944 having an internal thread 846 configured to cooperate with a corresponding thread of an insulin vial or container to couple the delivery module 900 to the vial container. The delivery module 900 is coupled to the insulin container via relative rotation of the connector body 902 and the insulin container. In embodiments, the penetrating tip 924 of the internal penetrating member 922 pierces a seal covering the outlet of the vial container to enable access of the administration member 918 to the insulin for distribution to the patient.

The delivery module 900 provides significant advantages over conventional delivery systems for delivering insulin. The internal penetrating member 922 eliminates the need for fabrication of a doubled end needle cannula or administration member 918. Piercing of the vial seal is effected through the internal penetrating tip 924. This also reduces the overall size of the administration member 918 relative to conventional delivery systems for insulin delivery. In embodiments, the reduction is size of the delivery cannula may range from a ten (10) % reduction to a fifty (50) percent reduction or about twenty-five (25) percent. In some embodiments, the length of the administration member may be about three (3) millimeters.

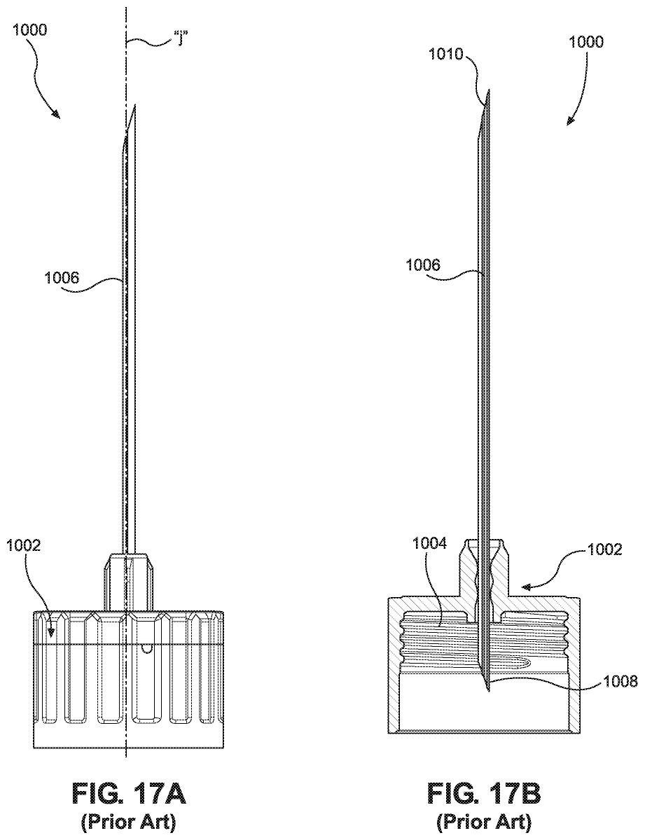

FIGS. 17A and 17B illustrate one conventional prior art delivery system for delivering insulin, the disadvantages of which are overcome by the delivery module 900 of FIGS. 16A and 16B. The conventional delivery assembly 1000 includes a housing 1002 with an internal thread 1004 and a needle 1006 coupled to the housing 1002. The needle 1006 is a double end needle having a sharp trailing end 1008 and a sharp leading end 1010. The needle 1006 is aligned with the central longitudinal axis “j” of the delivery module 900. The sharp trailing end 1008 is required to penetrate an insulin container to which the delivery assembly 1000 is coupled via the internal thread 1004. The necessity of a sharp trailing end 1008 adds additional steps during manufacture of the needle including grinding, polishing processes, which increases cost of manufacture. These steps are avoided with the delivery module 900 of FIGS. 16A and 16B via at least the internal penetrating member 922. In addition, due to the necessity of the sharpen trailing end 1008, the needle 1006 is relatively long, for example, at least twenty-five (25) percent longer than the administration member 918 of FIGS. 16A and 16B. In a typical arrangement, the needle 1006 may be about four (4) millimeters (mm) in length.

In some embodiments, the pre-filled medical delivery assemblies 10, 20, 30, 40, 50 may include a modular design consisting of separately constructed components cooperatively arranged and coupled to one another. According to some embodiments, the vial modules 100, 300, 400, 500, 700 and the delivery modules 200, 600, 800, 900 may be manufactured, packaged, shipped, stored, and/or provided as separate components. In such a manner, the delivery module 200, 600, 800, 900 may not need to be stored or shipped in accordance with often restrictive requirements imposed on medicaments and may accordingly reduce the amount of space required for such specialized storage and/or shipping. The delivery module 200, 600, 800, 900 may also or alternatively be manufactured, stored, and/or shipped in advance (e.g., at a first time) while the vial module 100, 300, 400 that is pre-filled with the fluid may be manufactured, stored, and/or shipped at a later time (e.g., a second time). In some embodiments, the delay between the first time and the second time may be lengthy without causing determinantal effects, as the delivery module 200 may be stored, in some embodiments, indefinitely. In such a manner, units of the delivery module 200, 600, 800, 900 may be provided to be on-hand in advance of the availability and/or arrival of the vial module 100, 300, 400, reducing supply chain constraints in the case of proactive the delivery module 200, 600, 800, 900 procurement.

### III. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention(s) nor a listing of features of the invention(s) that must be present in all embodiments.

Neither the Title (set forth at the beginning of the first page of this patent application) nor the Abstract (set forth at the end of this patent application) is to be taken as limiting in any way as the scope of the disclosed invention(s).

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

The phrase “coupled to”, when referring to two or more elements or features and their relationship to one another, is not intended to imply or infer that the two elements or features are separate units that are somehow attached, affixed, linked or configured to work together (although this may be true in some embodiments); rather, in at least some embodiment the two elements or features may be elements or features of a single larger element, component or body and may be molded or manufactured as a single element, component or body and/or be contiguously arranged such as to appear to a user to be two features or elements of a single component.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a step, method, or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the relevant embodiment, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise.

Headings of sections provided in this patent application and the title of this patent application are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining and the like.

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the relevant embodiment, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention(s) as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended points and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” This rule applies even within the body of a claim where a first instance of an element utilizes “a” or “an” and a second or subsequent instance of the element necessarily utilizes (e.g., for purposes of proper grammar and required antecedent basis) the definite article “the” to refer to the element. The use of the definite article “the” does not limit the element to a single object merely because it is utilized to refer back to a previous mention of the element. The original reference to the element controls with respect to the plurality (or lack thereof) of the element.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention(s) and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of the invention(s) in the various embodiments and equivalents thereof.

What Is Claimed Is:

1. A medical delivery apparatus for coupling to a container containing a fluid agent, the medical delivery apparatus comprising:
a delivery hub for coupling to a container containing a fluid agent, the delivery hub including:
a connector body for coupling to the container, the connector body defining a central longitudinal axis;
an administration hub segment for coupling with an administration member to enable distribution of the fluid agent from the container to a patient, the administration hub segment configured to arrange the administrator member about a first longitudinal axis; and
an internal penetrating member coupled to the connector body, the internal penetrating member including an internal penetrating tip configured to penetrate the container to access the fluid agent and arranged about a second longitudinal axis, the second longitudinal axis being different from the first longitudinal axis.

2. The medical delivery apparatus according to claim 1 wherein the administration hub segment defines a bore configured for reception of the administration member, the bore arranged about the first longitudinal axis.

3. The medical delivery apparatus according to claim 2 wherein the internal penetrating member defines an internal conduit in fluid communication with the administration member.

4. The medical delivery apparatus according to claim 3 wherein the internal conduit of the internal penetrating tip is in fluid coupled relation relative to the bore of administration hub segment.

5. The medical delivery apparatus according to claim 4 wherein the internal penetrating tip defines an inlet port in fluid communication with the internal conduit of the internal penetrating member.

6. The medical delivery apparatus according to claim 5 wherein the penetrating tip is configured to extend beyond the inlet port of the internal penetrating member.

7. The medical delivery apparatus according to claim 6 wherein the penetrating tip of the internal penetrating member is beveled.

8. The medical delivery apparatus according to claim 2 wherein the internal penetrating member is at least partially disposed within the connector body.

9. The medical delivery apparatus according to claim 2 wherein the internal penetrating member comprises a polymeric material.

10. The medical delivery apparatus according to claim 2 wherein the second longitudinal axis about which the internal penetrating member is arranged is in general alignment with the central longitudinal axis of the connector body.

11. The medical delivery apparatus according to claim 2 wherein inner surfaces of the administration hub segment defining the bore include one or more protrusions and one or more recesses.

12. The medical delivery apparatus according to claim 11 wherein the one or more protrusions are configured to engage the administration member.

13. The medical delivery apparatus according to claim 11 wherein the one or more recesses are configured to receive cement or glue used to secure the administration member within the bore.

14. The medical delivery apparatus according to claim 3 including the administration member secured within the bore of the administration hub segment.

15. The medical delivery apparatus according to claim 14 wherein the administration member comprises a needle defining a needle lumen in fluid communication with the internal conduit of the internal penetrating member.

16. The medical delivery apparatus according to claim 15 wherein the needle includes a leading penetrating needle tip.

17. The medical delivery apparatus according to claim 1 including a container coupled to the connector body of the delivery hub.

18. The medical delivery apparatus according to claim 17 wherein the container includes a vial having a vial body defining a reservoir for containing the fluid agent and a penetrable seal, the internal penetrating member configured to penetrate the penetrable seal when the vial is in coupled relation with the delivery hub.

19. A medical delivery system, comprising:
a vial module including a vial having a vial body defining a reservoir for containing a fluid agent and a penetrable seal; and
a delivery module including:
a connector body for coupling to the vial body, the connector body defining a central longitudinal axis;
an administration member coupled to the connector body and having a lumen to permit passage of the fluid agent from the vial to a patient, the administration member arranged about a first longitudinal axis; and
a penetrating member coupled to the connector body, the penetrating member defining an internal channel in fluid communication with the lumen of the administration member and having a penetrating tip, the penetrating member arranged about a second longitudinal axis, the second longitudinal axis radially offset relative to the first longitudinal axis of the administration member.

20. The medical delivery system according to claim 19 wherein the second longitudinal axis is in general alignment with the central longitudinal axis of the connector body.
